(19) United States
(12) Patent Application Publication
(10) Pub. No.: US 2018/0247011 A1
(43) Pub. Date: Aug. 30, 2018

(54) A METHOD FOR CD4+ T-CELL EPITOPE PREDICTION USING ANTIGEN STRUCTURE

(71) Applicant: THE ADMINISTRATORS OF THE TULANE EDUCATIONAL FUND, New Orleans, LA (US)

(72) Inventors: Ramgopal R. Mettu, New Orleans, LA (US); Samuel J. Landry, New Orleans, LA (US)

(21) Appl. No.: 15/756,372

(22) PCT Filed: Sep. 1, 2016
(86) PCT No.: PCT/US2016/049968

§ 371 Date: Feb. 28, 2018

Related U.S. Application Data
(60) Provisional application No. 62/212,827, filed on Sep. 1, 2015.

(51) Int. Cl. G06F 19/16; G06F 19/18; G06F 19/22; G06F 19/24; A61K 39/112; A61K 39/02

(52) U.S. Cl. CPC G06F 19/16; G06F 19/18; A61K 39/0208; G06F 19/24; A61K 39/0275; G06F 19/22

(57) ABSTRACT

The present invention relates to novel methods of diagnosing, preventing, and treating diseases, disorders, and infections relating to T-cell response. The disclosed methods for predicting MHC class II epitopes that elicit CD4+ T-cell response are N based on the three-dimensional protein structure of an antigen of interest. Given such an antigen, structural properties of the protein taken from experimental and modeling data are used to compute an epitope likelihood score that characterizes the location of epitopes likely to elicit an immune response to CD4+ T-cells. The epitopes are then used to construct biomolecules, including peptides, which may be used to diagnose, prevent, and/or treat a number of diseases, disorders, and infections.

### REFERENCE TO RELATED APPLICATIONS

This application claims priority to U.S. Provisional Application No. 62/212,827 filed Sep. 1, 2015, the complete disclosure of which is hereby fully incorporated herein by reference.

### STATEMENT REGARDING FEDERALLY SPONSORED RESEARCH

The invention was made with U.S. Government support from the National Science Foundation, grant no. NIH R01-A1080367, and the National Institutes of Health, grant no. NSF IIS-0643768. The United States Government has certain rights in the invention.

### FIELD OF THE INVENTION

The present invention relates to novel methods of identifying epitopes with enhanced MHC II binding characteristics and uses of such peptides in in preparing compositions for the treatment and diagnosis of diseases, disorders, and infections relating to T-cell response.

### BACKGROUND OF THE INVENTION

The major histocompatibility complex (MHC) molecules play a critical role in initiating immune response because they present antigen peptides on the cell surface for recognition by T cells. Endogenous proteins, such as self proteins, are processed in the cytosol and transported into the ER and loaded onto class I MHC (MHC-I) molecules. Exogenous proteins are taken up by endo/phagocytosis and processed into peptides and loaded onto MHC class II (MHC-II) molecules

MHC-I-peptide complexes bind to specific T-cell receptors on CD8 T-cells, which are cytotoxic, while MHC-II-peptide complexes bind T-cell receptors on CD4+ T-cells, which are more varied in nature. CD4+ T-cells provide numerous protective functions as part of the adaptive immune response, including cytokine-mediated and contact-mediated signals to B cells, CD8+ T-cells, and innate-immune cells, as well as direct modes of attack on pathogenic agents. While MHC-I and MHC-II molecules have multiple alleles in humans, the three-dimensional structure is highly conserved with allele variation occurring primarily in the peptide binding groove that influences antigen peptide specificity. The three-dimensional structures of both MHC-I and MHC-II molecules have been studied extensively. MHC-I molecules exhibit a highly sequence-specific preference for 9-mers in their closed binding grooves, while MHC-II molecules are less specific, with peptides being between 10 and 30 amino acids long.

Computational methods for predicting MHC-II-restricted epitopes are of great interest for understanding immune response to a variety of pathogens. The most accurate computational approaches to MHC binding prediction are currently based on modeling the sequence preferences for a given single MHC-I or MHC-II allele. While the particular machine learning method can vary, generally sequence-based methods work by using training data obtained from allele-specific MHC binding assays to construct a predictive model to predict a binding score. For example, the widely used NetMHC-II server performs MHC-II-restricted epitope prediction and utilizes a position-specific scoring matrix constructed from the training data for a specified allele.

Because MHC-I molecules are highly sequence specific and bind only 9-mers, supervised learning methods for epitope prediction have been successful due to the specificity of the MHC-I binding groove. Antigen processing and loading for the MHC-I pathway is more tightly orchestrated, with proteolysis occurring in one compartment and binding/loading occurring in another. The MHC-II presentation pathway is more challenging to model due to the open binding groove in the class II molecule, but also because antigen processing, loading, and binding happen concurrently. Recently, Wang et al. PLos Comput Biol (2008) 4:e1000048) showed that while a “consensus” approach to MHC binding prediction yields significant improvements in predictive power, these improvements do not necessarily carry over to the subsequent prediction of CD4+ T-cell immune response.

Early studies demonstrated that multiple lysosomal endoproteases and exoproteases participate in processing of antigens and that their activities were partially redundant. Processing steps are thought to occur both before and after peptide binding to the MHC protein. The elution of nested sets of peptides from naturally-loaded MHC proteins suggests that proteolytic trimming takes place after binding. However, other studies indicated that proteolysis must occur before binding. Watts and collaborators found that presentation of multiple T-cell epitopes in tetanus toxoid depended on an initial proteolytic cleavage by asparagine endoprotease. Presumably, the nicked protein was destabilized enough for unfolding to expose the epitopes for binding to the MHC-II protein. Disulfide crosslinks help a protein resist unfolding, and the works of Meric et al. Science (2001) 294:1361-65 and Nguyen et al. Vaccine (2015) 9:33:2887-96 demonstrated that disulfide bonds can block epitope immunogenicity. However, disulfide bonds can also have the opposite effect, to increase T-cell epitope immunogenicity, presumably by stabilizing the antigen against proteolytic destruction. On a more subtle level, dominant epitopes were reported to occur most frequently at sites adjacent to conformationally flexible protein segments, which may serve as entry points for proteolytic processing. Several studies have confirmed that these epitopes occur near the ends of peptides generated by limited proteolysis in vitro.

The intertwined mechanisms of antigen processing and peptide loading are further modulated by the action of HLA-DM and its regulator HLA-DO (or generically DM and DO). DM stimulates peptide exchange in MHC proteins, and mice that lack DM have altered epitope dominance patterns. DO inhibits DM by blocking the site that interacts with the MHC protein, and mice lacking DO also have altered epitope-specific responses. Efforts to specifically incorporate mechanisms of antigen processing or DM/DO-regulated peptide exchange for refinement of class II epitope prediction are not ongoing. One indirect effort utilized the SYFPEITHI database of natural MHC ligands to predict viral peptides that not only bind well to the MHC protein but also resemble the pools of natural (mostly self) ligands.

### SUMMARY OF THE INVENTION

In one example embodiment, a method for identifying immunogenic epitopes in target antigens comprises determining an epitope likelihood for reach residue in a target antigen based at least in art on a sequence and conformational stability profile for the target polypeptide and selecting one or more epitopes from the target antigen with an epitope likelihood score above a defined threshold. The epitope likelihood score may indicate a potential of a given epitope to bind MHC-II. Conformational stability profiles used to determine the epitope likelihood may include crystallographic B-factors, solvent-accessible area, and COREX residue stabilities.

In another example embodiment, a method for identifying immunogenic epitopes in target antigens may comprise identifying one or more epitopes within a target antigen using one or more classifiers applied to the antigen sequence and a conformational stability profile. In certain example embodiments, the classifier is derived using a hidden markov model (HMM) machine learning method or a position-specific scoring matrix (PSSM) model. Conformational stability profiles used to determine the epitope likelihood may include crystallographic B-factors, solvent-accessible area, and COREX residue stabilities. In certain example embodiments, the classifiers are derived by training the classifiers on peptide sequences known to bind to MHC II complexes along with respective conformational stability data as derived from the parent antigen.

In another embodiment, the methods may further comprise preparing one or more compositions based on the epitopes identified as described herein. The peptide epitopes may be formulated as use for diagnostics or therapeutics. In certain example embodiments, the methods further comprise administration of said compositions for the purposes of treating various diseases and disorders associated with an immune response including, but not limited to, autoimmune disease, pathogenic infections, and cancer, or administration of said epitopes as diagnostics for identifying the presence of immune system components associated with certain diseases and disorders.

These and other aspects, objects, features, and advantages of the example embodiments will become apparent to those having ordinary skill in the art upon consideration of the following detailed description.

### BRIEF DESCRIPTION OF THE DRAWINGS

FIG. 1: Comparison of single-allele prediction accuracy.

FIG. 2: Receiver-operator curves for single allele predictions.

FIG. 3: Prediction accuracy for human data.

FIG. 4: LcrV predictions.

FIG. 5: Anthrax protective antigen predictions. Conformational stability data is shown by residue (a), and the resulting epitope likelihood score for sequential 20-mers is shown in (b). For comparison, MHC binding scores are shown in (d). Corresponding 90th and 80th percentile thresholds are shown as a red dashed line for sorted scores (c, e).

FIG. 6: Epitope Frequency for Antigen Systems. In C57BL/6 mice, the various antigens primed as few as two and as many as eleven epitopes, which were discovered by testing with peptide sets of 20-89 peptides. In general, the number of epitopes correlated with the size of the antigen, with an epitope appearing on-average every 58 residues. This density of epitopes is similar to that previously reported for a collection of nine antigens and allergens. For the collection of 7 antigens in C57BL/6 mice, the rate of epitope discovery is the number of epitopes divided by the number of test peptides. Expressed as a percentage, the rate was 15%. This rate imposes a lower limit on the accuracy of epitope prediction because 15% of peptides randomly selected from the test set are expected to contain an epitope.

FIG. 7: Birch Bet v predictions. Conformational stability data is shown by residue (a). The computed epitope likelihood score for sequential 12-mers is shown in (b), along with a sorted view of the scores with 90th and 80th percentile thresholds (red dashed lines).

FIG. 8: Adenovirus type 5 hexon predictions. Conformational stability data is shown by residue (a). The computed epitope likelihood score for sequential 15-mers is shown in (b), along with a sorted view of the scores with 90th and 80th percentile thresholds (dashed lines).

FIG. 9: (a) Overall accuracy of prediction at the 80th percentile threshold of the random baseline and stability-based prediction. (b) ROC curve for stability-based prediction.

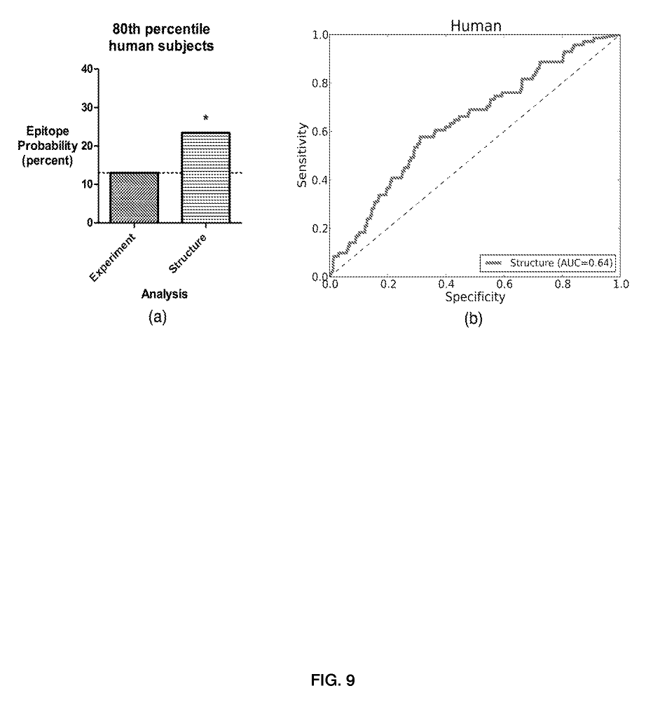

FIG. 10 is a block flow diagram depicting a method for identifying immunogenic epitopes in target antigens in accordance with certain example embodiments.

FIG. 11 is a block flow diagram depicting a method for identifying immunogenic epitopes in target antigens in accordance with certain example embodiments.

FIG. 12 is a set of graphs demonstrating epitope prediction performance for a set of 16 human proteins expressed in bone marrow and commonly used in cancer research, in accordance with certain example embodiments.

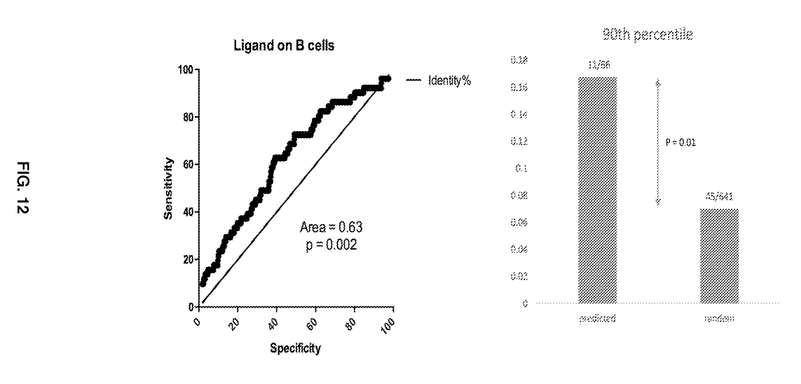

FIG. 13 are a series of graph showing predictive performance of methods disclosed herein of a common antigen across four viruses (Dengue fever, yellow fever, tick-borne encephalitis, Japanese encephalitis). Results are show across the bottom row (“post-fusion”). The plots how the prediction score (dark gray, diagonal) and epitopes as a vertical line. The texts shows how many of the experimental epitopes are captured for each antigen when the top 80% scoring peptides are considered.

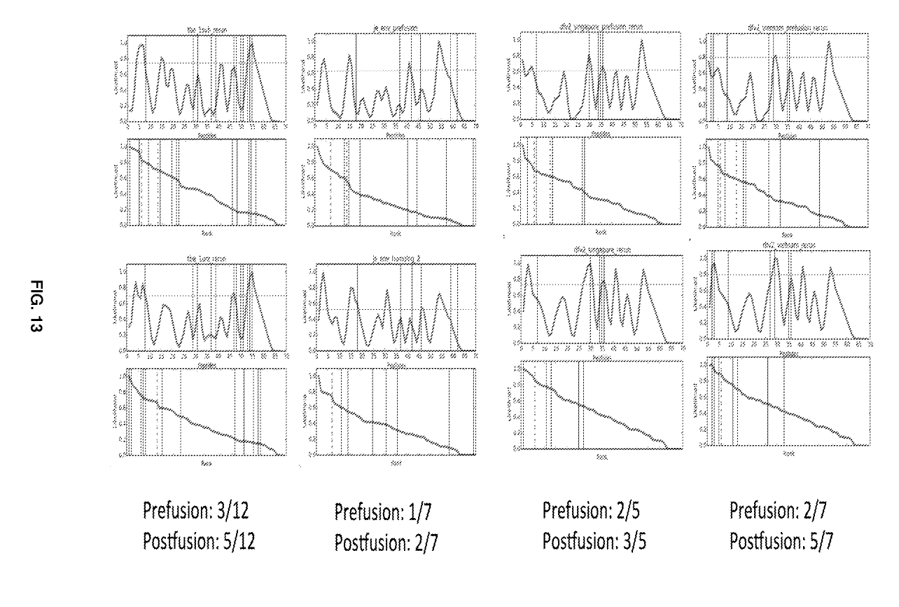

### DETAILED DESCRIPTION OF THE INVENTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Wherever any of the phrases “for example,” “such as,” “including” and the like are used herein, the phrase “and without limitation” is understood to follow unless explicitly stated otherwise. Similarly “example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “epitope” refers to short peptide sequences derived from larger sequences of a target antigen that may be recognized by antibodies, MHC II complexes, or other cell surface receptors of immune cells.

### OVERVIEW

The present invention discloses methods for MHC-II-restricted epitope prediction that utilize peptide sequence and conformational stability data of a given antigen. Data presented herein demonstrate that mechanisms for antigen processing, and thus antigen three-dimensional structure, guide the ultimate presentation of an epitope. The method disclosed by the present invention uses experimental and predicted conformational stability criteria to compute an epitope likelihood score for any peptide in the antigen sequence. The method disclosed herein has significant advantages over existing MHC-binding based prediction schemes, which have relatively weak predictive power for the task of epitope prediction. In multiple-allele systems, the present invention provides a means of epitope prediction that does not rely on a priori knowledge of MHC allele distribution, which is of considerable use in a number of applications such as the design of therapeutics, vaccines, and diagnostics.

CD4+ T cells play multiple critical, yet poorly understood roles in immunity. CD4+ T cells are primed through multiple epitope-specific cell-cell contacts, starting with an antigen-presenting dendritic cell. Subsequently, mature CD4+ T cells arouse or deliver immune defense mechanisms when they recognize their epitopes, as presented by dendritic cells, or B cells, or other antigen-presenting cells, including macrophages and epithelial cells. Most antigens are taken up by endocytic mechanisms and undergo proteolytic processing within endocytic compartments, ultimately yielding a family of approximately 15 residue-long peptides in complexes with the class II major histocompatibility proteins (MHCII). As presented by the MHC-II, the peptides provide the antigen-specific features of the epitopes recognized by the T cells. The lack of epitope-specific reagents presents a major barrier to study of CD4+ T-cells.

Previously, CD4+ T-cell epitope prediction was based solely on the affinity of antigen sequences for the MHC-II protein. However, that approach has been plagued with a very high false-positive rate, and it does not take into account the variable concentration of proteolytic fragments generated by antigen processing. It is possible that the false positives correspond to epitopes due to preferential accumulation of proteolytic fragments.

Embodiments disclosed herein provide an approach to CD4+ T-cell epitope prediction that utilizes antigen conformational stability rather than MHC-II binding affinity. Mettu et al. Journal Immunol Methods (2016), 432:72-81. The methods disclosed herein provide results comparable, if not improved over, existing epitope prediction methodologies for single-allele systems, and provides significantly increased predictive power for multiple-allele systems without requiring knowledge of a subject's HLA genotypes.

Certain aspects of the embodiments disclosed herein may be implemented on a computer-based system. The system may comprises a user computing device comprising a user interface application for uploading sequence and conformational stability data for target antigens to be analyzed, as well as review outputs of the epitope analysis such as graphs of epitope likelihood scores and/or epitope maps showing the location of epitopes identified according to the methods disclosed herein and/or peptide sequences of the epitopes disclosed herein. The computing device may further include a epitope discovery module comprising computer-executable program instructions embodied on a non-transitory computer-readable storage device of the computing device to carry out the methods disclosed herein. The computing device may further include a wired or wireless telecommunications means by which the computing device can exchange data with one or more additional computing devices. For example, sequence and conformational stability data may be stored on one or more external servers and accessed from the computing device when implementing the steps of the embodiments disclosed herein. Accordingly, each computing device may include a communication module capable of transmitting and receiving data over a network, such as local area network (“LAN”), a wide area network (“WAN”), an intranet, an Internet, a mobile telephone network, or any combination thereof. Throughout the discussion of example embodiments, it should be understood that the terms “data” and “information” are used interchangeably herein to refer to text, images, audio, video, or any other form of information that can exist in a computer-based environment. The computing device may include a server, desktop computer, laptop computer, tablet computer, smart phone, or any other wired or wireless, processor-driven device. In certain example embodiments, the computing device may be networked directly to a protein sequencing device—such as but not limited to a mass spectroscopy device—and/or a protein 3D structure device—such as but no limited to an X-ray crystallography, NMR spectroscopy or electron microscopy device—in order to obtain direct sequence and/or structure data of an antigen or antigens of interest. In other example embodiments, the antigen sequence and structural data may be obtained from one or more other computing devices or uploaded directly to the computing device executing the methods disclosed herein.

Turning now to the drawings, in which like numerals represent like (but not necessarily identical) elements throughout the figures, example embodiments are described in detail.

### Example Processes

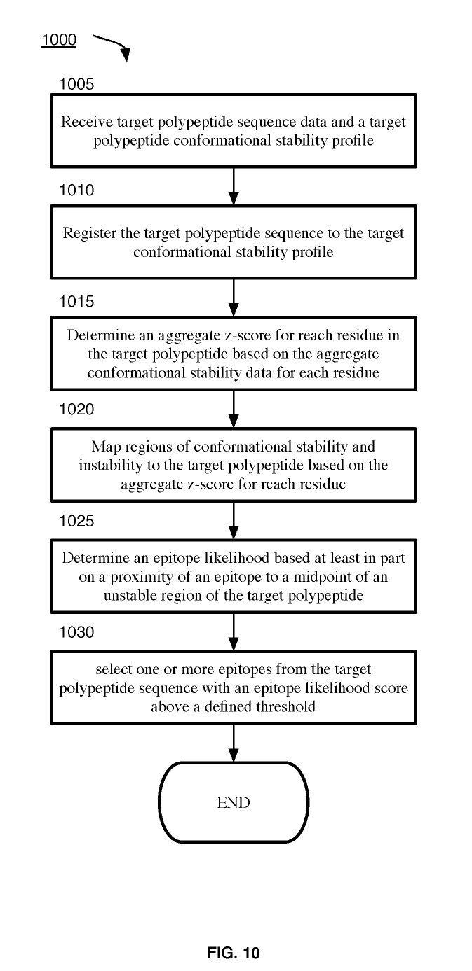

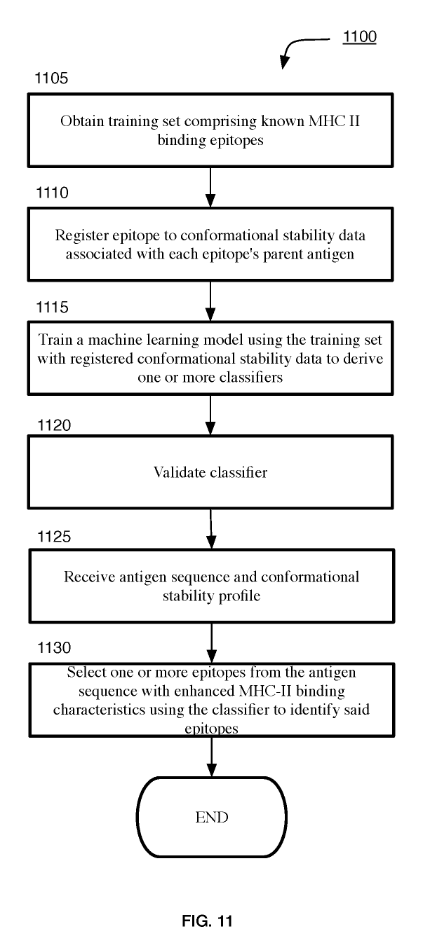

The example illustrated in FIGS. 10 and 11 are described with reference to the computing devices and computing environment described herein. The example method of FIGS. 10 and 11 may also be performed with other systems and in other environments.

FIG. 10 is a block flow diagram depicting a method 1000, which provides an example process for identifying epitopes having enhanced MHC-II binding characteristics.

Method 1000 begins at block 1005, where an epitope discovery module receives sequence data for a target antigen and a corresponding conformational stability profile for that antigen. In certain example embodiments, multiple antigen sequences and corresponding conformational stability profiles may be uploaded simultaneously in a batch mode. The sequence data may be uploaded from a database comprising protein sequence data for the antigen or antigens of interests. Alternatively, the antigen peptide sequence data may be obtained directly from peptide sequencing of the antigen. For example, the peptide sequence may come from sequencing of an antigen or set of antigens by a peptide sequencing device. Likewise, the corresponding conformational stability profiles may be obtained from a database or directly from a 3D structural imaging device. In certain example embodiments, the conformational stability profile may comprise a protein data bank (PDB) file, wherein the PDB file comprises one or more types of conformational data for each residue in the target antigen. In certain example embodiments, the PDB file comprises one or more of crystallographic B-factors, solvent accessible area, and COREX residue stability data for reach residue in an antigen.

At block 1010, the epitope discovery module registers the antigen sequence data to the conformational stability profile such that each residue in the antigen sequence is associated with a set of conformational stability data for that residue. In certain example embodiments, one or more of crystallographic B-factors, solvent accessible area, and COREX residue stability data is associated with each corresponding residue. In certain example embodiments, certain residues in the target antigen may be missing conformational stability data. In certain example embodiments, such gaps are addressed by assigning default values for each type of conformational stability data type that indicate a minimum level of stability. For example, a missing B-factor would be set to the maximum B-factor observed in the conformational stability data over all residues in that antigen. In certain example embodiments, the data may be smoothed by taking a windowed averaged across each dataset. In certain example embodiments, the window size is 15 residues.

At block 1015, the epitope discovery module determines an aggregate z-score for each residue based on the aggregate conformational stability data for reach residue. In certain example embodiments, this may be done by first determining z-scores for every residue with respect to each dataset, viewing each type of conformational data as an independent observation of antigen conformational stability. Then, the individual z-scores for reach residue are combined to determine the aggregate z-score. In certain example embodiments, this may be done by using an analog of Fisher's method for combining test statistics. The aggregate z-score characterizes the extent to which the structural data agree that the antigen is conformationaly unstable at a given position. In certain example embodiments, any residue with an aggregate z-score greater than zero is stable and all other residues are considered unstable.

At block 1020, the epitope discovery module maps the regions of conformational stability and instability to the target antigen based on the aggregate z-score determined for each residue. For example, the epitope discovery module may record a start and end residue for a series of residues that are unstable or stable as determined by the aggregate z-score associated with each residue.

At block 1025, the epitope discovery module determines an epitope likelihood based at least in part on a proximity of an residue or set of residues to a midpoint of an unstable region of the target antigen. In certain example embodiments, this is done by setting an epitope likelihood score at each residue in an unstable region to zero. Stable residues adopt the previously determined aggregate z-score as the epitope likelihood score. Epitope likelihoods in regions of the antigen that transition from unstable to stable, or vice versa, are upweighted. For example, consider an unstable region and the C-terminally adjacent stable region. First, the epitope likelihoods for the first third of the stable region are upweighted by a factor of three. Then epitope likelihoods are assigned by linearly interpolating from a midpoint of the unstable region to the midpoint of the upweighted portion of the C-terminal flanking stable region. This same unweighting can be applied to the N-terminal flanking region or to both the C-terminal and N-terminal flanks of an unstable region. In certain example embodiments, multiple allele systems are upweighted in both the C-terminal and N-terminal flanking regions.

At block 1030, the epitope discovery module selects one or more epitopes that are above a defined epitope likelihood threshold. In certain example embodiments, only peptides with an epitope score greater than 60%, 65%, 70%, 75%, 80%, 85%, 90%, or 95% are selected by the methods disclosed herein. In certain example embodiments, only peptides with an epitope likelihood score of 80% are selected. In certain other example embodiments, only peptides with an epitope likelihood score of 90% are selected.

In certain embodiments, machine learning is used in place of, or in conjunction with the above described process to identify epitopes in target antigens with enhanced MCH-II binding characteristics. In certain example embodiments, supervised machine learning is applied to a training data set to derive one or more classifiers that can take target antigen sequences and corresponding conformational stability profiles as input and identify epitopes within the antigen that have enhanced MCH-II binding characteristics.

Turning now to FIG. 11, which describes an example process 1100 for identifying immunogenic epitopes in target antigens. Process 1100 begins at block 1105, where the epitope discovery module obtains a training set of epitope sequences known to bind MHC-II complexes. The epitope sequence data may be obtained experimentally using known methods in the art, or as further described in the Examples section below. Likewise, the epitope sequence information may be obtained from databases containing such information. An example database for epitope information includes, but is not limited to, the Immune Epitope Database (IEDB), which contains peptides with T-cell response data for available single and multi-allele systems. In addition, the epitope discovery module either receives corresponding conformational stability data for each antigen or obtains this information from a separate database based on one or more identifiers associated with the epitope. For example, metadata associated with the epitope sequences may reference one or more identifiers of the parent antigen that allow the epitope discovery module to pull corresponding conformational stability information associated with the parent antigen in a separate protein database, such as the Protein Data Bank database.

At block 1110, the epitope discovery module registers the conformational stability data to reach residue from the parent antigen. In situations where the epitope sequence position within the parent antigen is not known, the epitope discovery module may first execute an alignment with the sequence of the parent antigen using alignment methods known in the art. Otherwise, the epitope discovery module will use location information associated with the epitope sequence to pull the corresponding conformational stability data from the parent antigen conformational stability profile and associate it with each residue of the epitope. In certain example embodiments, the types of conformational stability data associated with each epitope residue may comprise crystallographic B-factors, solvent accessible area, and COREX residue stability data.

At block 115, the epitope discovery module applies the registered epitope sequence and conformational stability data to a supervised machine learning model. In certain example embodiments, the machine learning model is a hidden Markov model (HMM). HMMs only require sequential dependencies between amino acid positions across the training set and are computationally efficient to train. In certain other example embodiments, a position-specific scoring matrix (PSSM) model is used. PSSMs construct an explicit sequence motif, but require multiple sequence alignments on all peptides in the training set. In certain example embodiments structural data is incorporated into a Gibbs sampling approach. Nielsen et al. BMC Bioinformatics, (2007) 8:238. In one example embodiment, peptide contributions to the final motif are pre-weighted utilizing an aggregate measure of conformational stability per peptide in the training set. The conformational stability data is then incorporated into a Gibbs sampler so as to bias the search for the motif in a structurally appropriate manner. In addition, “pseudocounts” are applied, which are correction factors used to smooth or normalize amino acid frequencies that occur in the motif as it is being constructed.

At block 1120, the epitope discovery module validates the one or more classifiers. For example, the training set may be split into a training set and a test set, with the training set used for training purposes as described above, and the test stet used to validate the efficiency and or accuracy of the derived classifier. Validation may be conducted according to known methods in the art and as further outlined in the Example section below.

Once a classifier or classifiers are derived using process 1100, future epitopes may be predicted by providing antigen sequence information and conformational stability profiles. The classifier may then identify epitopes with enhanced MHC II binding characteristics, or may score each epitope with a score indicating a likelihood that each identified epitope will bind to a MHC II complex. For example, at block 1125, the epitope discovery module receives antigen sequence and corresponding conformational stability data for a target antigen or antigens as described previously.

At block 1130, the epitope discovery module applies the inputs to the trained HMM or PSSM model which outputs one or more epitopes with enhanced MEW II binding characteristics. In certain example embodiments, the trained HMM or PSSM model may classify a given epitope as having MEW II binding characteristics. In certain other example embodiments, the trained HMM or PSSM may provide a score, such as between 0 and 1 (0% and 100%), indicating the likelihood of a given epitope to bind a MHC-II complex.

### Therapeutic and Diagnostic Applications

The above methods may used to develop compositions for diagnostic and therapeutic uses, including therapeutic vaccine design by identifying those epitopes most likely to bind to MEW II and elicit an immune response. Accordingly, the methods disclosed herein may be used to improve the efficiency and efficacy of peptide and vaccine therapeutic design. Thus, in certain example embodiments, the method may further comprise preparing one or more compositions comprising one or more epitopes identified using the above methods. The epitope peptides may be synthesized de novo using known chemical or molecular biology techniques or otherwise isolated from an existing antigen population.

In a preferred embodiment, the at least one biomolecule is comprised of a Class II MEW molecule fused or bound to one or more peptides.

The epitope peptides described herein can be provided as physiologically acceptable formulations using known techniques. Remington's Pharmaceutical Sciences, by E. W. Martin, Mack Publishing Co., Easton, Pa., 19th Edition (1995), describes compositions and formulations suitable for pharmaceutical delivery of peptides disclosed herein.

The formulations in accordance with the present invention can be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, an implant, in a device, as an eye drop or a transdermal patch.

The formulations include those suitable for oral, rectal, nasal, inhalation, topical (including dermal, transdermal, buccal, and eye drops), vaginal, parenteral (including subcutaneous, intramuscular, intravenous, intradermal, intraocular, intratracheal, and epidural), ophthalmic (periocular, intraocular, including suprachoroidal, subretinal, and intravitreal), or inhalation administration. In one exemplary embodiment, the peptides of the present invention are formulated for transcleral, suprachoroidal, subretinal, or intravitreal delivery. Transcleral delivery includes subconjunctival, subtenons′, and retrobulbar transcleral delivery. The formulations can conveniently be presented in unit dosage form and can be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored base, usually sucrose and acacia or tragacanth; pastilles comprising the active ingredient in an inert base such as gelatin and glycerin, or sucrose and acacia; and mouthwashes comprising the ingredient to be administered in a suitable liquid carrier.

Formulations suitable for topical administration to the skin may be presented as ointments, creams, gels, pastes, and eye drops comprising the ingredient to be administered in a pharmaceutical acceptable carrier.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents; gels; and surgically placed implants.

In some embodiments, the disorder to be treated or prevented is an autoimmune disorder. The autoimmune disorder may be selected from the group comprising rheumatoid arthritis, osteoarthritis, lupus, Addison's disease, Celiac disease, Dermatomyositis, Graves' disease, Hashimoto's thyroiditis, Multiple sclerosis, Myasthenia gravis, Pernicious anemia, Reactive arthritis, Rheumatoid arthritis, Sjogren syndrome, Systemic lupus erythematosus, and Type I diabetes. In other embodiments, the disease to be treated or prevented is human immunodeficiency virus infection (HIV) and/or acquired immune deficiency syndrome (AIDS). In some embodiments, the disease to be treated or prevented is cancer. In other embodiments, the infection to be treated or prevented may be viral, bacterial, protozoan, or fungal, and it may be chronic or acute. In some embodiments, the disorder to be treated or prevented is an allergic reaction, including any allergic reactions to food, insect sting, pollen, dust mites, mold, fungus, animal dander, cockroach, and protein-based drugs.

In another embodiment, the present invention discloses a method of diagnosing a disease, disorder, or infection in a subject comprising identifying at least one target antigen causing said disease, disorder, or infection; collecting sequence data and conformational stability data for the at least one antigen; computing an epitope likelihood from said antigen sequence data and conformational stability data for each antigen residue; locating an epitope of said at least one target antigen by mapping the epitope likelihood and selecting the peaks above a predetermined level; constructing at least one biomolecule that contains one or more epitopes of the at least one target antigen; obtaining a blood sample from said subject; combining said blood sample with an effective amount of a composition comprising said biomolecule; and determining whether said blood sample contains said at least one antigen causing said disease, disorder, or infection. In some embodiments, the subject is a mammal. In some embodiments, the mammal is a human.

In some embodiments, the disorder to be diagnosed is an autoimmune disorder. The autoimmune disorder may be selected from the group comprising rheumatoid arthritis, osteoarthritis, lupus, Addison's disease, Celiac disease, Dermatomyositis, Graves' disease, Hashimoto's thyroiditis, Multiple sclerosis, Myasthenia gravis, Pernicious anemia, Reactive arthritis, Rheumatoid arthritis, Sjogren syndrome, Systemic lupus erythematosus, and Type I diabetes. In other embodiments, the disease to be diagnosed is human immunodeficiency virus infection (HIV) and/or acquired immune deficiency syndrome (AIDS). In other embodiments, the disease to be diagnosed is cancer. In other embodiments, the infection to be diagnosed may be viral, bacterial, protozoan, or fungal, and it may be chronic or acute. In other embodiments, the disorder to be diagnosed is an allergic reaction, including any allergic reactions to food, insect sting, pollen, dust mites, mold, fungus, animal dander, cockroach, and protein-based drugs.

In another embodiment, the present invention provides a method of diagnosing a disease, disorder, or infection in a subject comprising identifying at least one target antigen causing said disease, disorder, or infection; collecting sequence data and conformational stability data for the at least one antigen; computing an epitope likelihood from said antigen sequence data and conformational stability data for each antigen residue; locating an epitope of said at least one target antigen by mapping the epitope likelihood and selecting the peaks above a predetermined level; constructing at least one biomolecule that contains one or more epitopes of the at least one target antigen; constructing at least one Class II MHC tetramer comprising the at least one biomolecule and at least one tag; obtaining a blood sample from said subject; combining said blood sample with an effective amount of the at least one Class II MHC tetramer; and determining whether said blood sample contains said at least one antigen causing said disease, disorder, or infection. In some embodiments, the subject is a mammal. In some embodiments, the mammal is a human.

In a preferred embodiment, the at least one biomolecule is comprised of a Class II MHC molecule fused or bound to one or more peptides. In a preferred embodiment, flow cytometry is used to determine whether said blood sample contains said at least one antigen causing said disease, disorder, or infection. wherein said antigen sequence data and conformation stability data comprises crystallographic B-factor data, solvent-accessible surface area data, COREX residue stabilities data, and/or sequence conservation data. In some embodiments, the predetermined level comprises at least the 80th percentile, and in some embodiments, the predetermined level comprises at least the 90th percentile.

In some embodiments, the disorder to be diagnosed is an autoimmune disorder. The autoimmune disorder may be selected from the group comprising rheumatoid arthritis, osteoarthritis, lupus, Addison's disease, Celiac disease, Dermatomyositis, Graves' disease, Hashimoto's thyroiditis, Multiple sclerosis, Myasthenia gravis, Pernicious anemia, Reactive arthritis, Rheumatoid arthritis, Sjogren syndrome, Systemic lupus erythematosus, and Type I diabetes. In other embodiments, the disease to be diagnosed is human immunodeficiency virus infection (HIV) and/or acquired immune deficiency syndrome (AIDS). In some embodiments, the disease to be diagnosed is cancer. In some embodiments, the infection to be diagnosed may be viral, bacterial, protozoan, or fungal, and the infection may be chronic or acute. In other embodiments, the disorder is an allergic reaction, including any allergic reactions to food, insect sting, pollen, dust mites, mold, fungus, animal dander, cockroach, and protein-based drugs.

In one embodiment, the present invention discloses a biomolecule produced by any of the methods described above. In a preferred embodiment, the at least one biomolecule is comprised of a Class II MHC molecule fused or bound to a peptide. In some embodiments, the present invention provides a pharmaceutical composition comprising a biomolecule produced by any of the methods described above. In some embodiments, the pharmaceutical composition may further comprise an adjuvant. In some embodiments, the present invention discloses a Class II MHC tetramer produced by any of the methods described above. In a preferred invention, the at least one biomolecule included in said tetramer is a peptide. In some embodiments, the at least one tag included in said tetramer is a fluorescent tag.

In another embodiment, the present invention provides a kit for the diagnosis of a disorder, disease, or infection comprising at least one biomolecule produced by any of the methods; and a substrate to allow for combining said at least one biomolecule with a blood sample from a patient.

In another embodiment, the present invention discloses a kit for the diagnosis of a disorder, disease, or infection, comprising at least one Class II MHC tetramer produced by the any of the methods described above and a substrate to allow for combining the at least one Class II MHC tetramer with a blood sample from a subject. In some embodiments, the present invention provides a kit for the diagnosis of a disorder, disease, or infection, comprising at least one Class II MHC molecule; at least one biomolecule produced by any of the methods described above; at least one tag; and a substrate to allow for combining the at least one Class II MHC tetramer with a blood sample from a subject.

The invention is further described with reference to the following numbered paragraphs.

1. A method of preventing or treating a disease, disorder, or infection in a subject comprising:
a. identifying at least one target antigen causing said disease, disorder, or infection;
b. collecting sequence data and conformational stability data for such at least one antigen;
c. computing an epitope likelihood from said antigen sequence data and conformational stability data for each antigen residue;
d. locating an epitope of said target antigen by mapping the epitope likelihood and selecting the peaks above a predetermined level;
e. constructing at least one biomolecule that contains one or more epitopes of the target antigen;
f. administering to the subject an effective amount of a composition comprising said at least one biomolecule; and
g. determining that the development of the disease, disorder, or infection has been prevented, minimized, or reversed.

2. The method of claim 1, wherein said at least one biomolecule is one or more peptides.

3. The method of claim 1, wherein said composition further comprises one or more adjuvants.

4. The method of claim 1, wherein said antigen sequence data and conformation stability data comprises crystallographic B-factor data, solvent-accessible surface area data, COREX residue stabilities data, and/or sequence conservation data.

5. The method of claim 1, wherein the predetermined level comprises at least the 80^{th} percentile.

6. The method of claim 1, wherein the predetermined level comprises at least the 90^{th} percentile.

7. The method of claim 1, wherein said composition is in a form of a product for oral delivery, said product from being selected from a group consisting of a concentrate, a dried powder, a liquid, a capsule, a pellet, or a pill.

8. The method of claim 1 wherein said composition is in a form of a product for parenteral administration including intravenous, intradermal, intramuscular, and subcutaneous administration.

9. The method of claim 1 wherein said composition further comprises at least one carrier, at least one binder, at least one diluent, at least one excipient, or mixtures thereof.

10. The method of claim 1, wherein the disorder is an autoimmune disorder.

11. The method of claim 11, wherein the autoimmune disorder is selected from the group comprising rheumatoid arthritis, osteoarthritis, lupus, Addison's disease, Celiac disease, Dermatomyositis, Graves' disease, Hashimoto's thyroiditis, Multiple sclerosis, Myasthenia gravis, Pernicious anemia, Reactive arthritis, Rheumatoid arthritis, Sjogren syndrome, Systemic lupus erythematosus, and Type I diabetes.

12. The method of claim 1, wherein the disease is selected from the group comprising human immunodeficiency virus (HIV) and acquired immune deficiency syndrome (AIDS.)

13. The method of claim 1, wherein the disease is cancer.

14. The method of claim 1, wherein the infection is viral, bacterial, protozoan, or fungal.

15. The method of claim 14, wherein the infection is chronic or acute.

16. The method of claim 1, wherein the disorder is an allergic reaction.

17. The method of claim 16, wherein the allergic reaction is selected from the group comprising reaction to food, insect sting, pollen, dust mites, mold, fungus, animal dander, cockroach, and protein-based drugs.

18. The method of claim 1, wherein the subject is a mammal.

19. A method of diagnosing a disease, disorder, or infection in a subject comprising:
a. identifying at least one target antigen causing said disease, disorder, or infection;
b. collecting sequence data and conformational stability data for the at least one antigen;
c. computing an epitope likelihood from said antigen sequence data and conformational stability data for each antigen residue;
d. locating an epitope of said at least one target antigen by mapping the epitope likelihood and selecting the peaks above a predetermined level;
e. constructing at least one biomolecule that contains one or more epitopes of the at least one target antigen;
f. obtaining a blood sample from said subject;
g. combining said blood sample with an effective amount of a composition comprising said biomolecule; and
h. determining whether said blood sample contains said at least one antigen causing said disease, disorder, or infection.

20. The method of claim 19, wherein the at least one biomolecule is one or more peptides.

21. The method of claim 19, wherein said antigen sequence data and conformation stability data comprises crystallographic B-factor data, solvent-accessible surface area data, COREX residue stabilities data, and/or sequence conservation data.

22. The method of claim 19, wherein the predetermined level comprises at least the 80^{th} percentile.

23. The method of claim 19, wherein the predetermined level comprises at least the 90^{th} percentile.

24. The method of claim 19, wherein the disorder is an autoimmune disorder.

25. The method of claim 24, wherein the autoimmune disorder is selected from the group comprising rheumatoid arthritis, osteoarthritis, lupus, Addison's disease, Celiac disease, Dermatomyositis, Graves' disease, Hashimoto's thyroiditis, Multiple sclerosis, Myasthenia gravis, Pernicious anemia, Reactive arthritis, Rheumatoid arthritis, Sjogren syndrome, Systemic lupus erythematosus, and Type I diabetes.

26. The method of claim 19, wherein the disease is selected from the group comprising human immunodeficiency virus (HIV) and acquired immune deficiency syndrome (AIDS.)

27. The method of claim 19, wherein the disease is cancer.

28. The method of claim 19, wherein the infection is viral, bacterial, protozoan, or fungal.

29. The method of claim 19, wherein the infection is chronic or acute.

30. The method of claim 19, wherein the disorder is an allergic reaction.

31. The method of claim 30, wherein the allergic reaction is selected from the group comprising reaction to food, insect sting, pollen, dust mites, mold, fungus, animal dander, cockroach, and protein-based drugs.

32. The method of claim 19, wherein the subject is a mammal.

33. A method of diagnosing a disease, disorder, or infection in a subject comprising:
a. identifying at least one target antigen causing said disease, disorder, or infection;
b. collecting sequence data and conformational stability data for the at least one antigen;
c. computing an epitope likelihood from said antigen sequence data and conformational stability data for each antigen residue;
d. locating an epitope of said at least one target antigen by mapping the epitope likelihood and selecting the peaks above a predetermined level;
e. constructing at least one biomolecule that contains one or more epitopes of the at least one target antigen;
f. constructing at least one Class II MHC tetramer comprising the at least one biomolecule and at least one tag;
g. obtaining a blood sample from said subject;
h. combining said blood sample with an effective amount of the at least one Class II MHC tetramer; and
i. determining whether said blood sample contains said at least one antigen causing said disease, disorder, or infection.

34. The method of claim 33, wherein the at least one biomolecule is one or more peptides.

35. The method of claim 33, wherein flow cytometry is used to determine whether said blood sample contains said at least one antigen causing said disease, disorder, or infection.

36. The method of claim 33, wherein said antigen sequence data and conformation stability data comprises crystallographic B-factor data, solvent-accessible surface area data, COREX residue stabilities data, and/or sequence conservation data.

37. The method of claim 33, wherein the predetermined level comprises at least the 80^{th} percentile.

38. The method of claim 33, wherein the predetermined level comprises at least the 90^{th} percentile.

39. The method of claim 33, wherein the disorder is an autoimmune disorder.

40. The method of claim 39, wherein the autoimmune disorder is selected from the group comprising rheumatoid arthritis, osteoarthritis, lupus, Addison's disease, Celiac disease, Dermatomyositis, Graves' disease, Hashimoto's thyroiditis, Multiple sclerosis, Myasthenia gravis, Pernicious anemia, Reactive arthritis, Rheumatoid arthritis, Sjogren syndrome, Systemic lupus erythematosus, and Type I diabetes.

41. The method of claim 33, wherein the disease is selected from the group comprising human immunodeficiency virus (HIV) and acquired immune deficiency virus (AIDS.)

42. The method of claim 33, wherein the disease is cancer.

43. The method of claim 33, wherein the infection is viral, bacterial, protozoan, or fungal.

44. The method of claim 33, wherein the infection is chronic or acute.

45. The method of claim 33, wherein the disorder is an allergic reaction.

46. The method of claim 45, wherein the allergic reaction is selected from the group comprising reaction to food, insect sting, pollen, dust mites, mold, fungus, animal dander, cockroach, and protein-based drugs.

47. The method of claim 33, wherein the subject is a mammal.

48. A biomolecule produced by the method of claim 1.

49. The biomolecule of claim 48, wherein the biomolecule is a peptide.

50. A pharmaceutical composition comprising a biomolecule produced by the method of claim 1.

51. The pharmaceutical composition of claim 49, further comprising an adjuvant.

52. A biomolecule produced by the method of claim 19.

53. The biomolecule of claim 52, wherein the biomolecule is a peptide.

54. A biomolecule produced by the method of claim 33.

55. The biomolecule of claim 54, wherein the biomolecule is a peptide.

56. A Class II WIC tetramer produced by the method of claim 33.

57. The tetramer of claim 56, wherein the at least one biomolecule is a peptide.

58. The tetramer of claim 56, wherein at least one biomolecule is comprised of the WIC II molecule fused to a peptide or bound with a peptide.

59. The tetramer of claim 56, wherein the at least one tag is a fluorescent tag.

60. A kit for the diagnosis of a disorder, disease, or infection comprising
a. at least one biomolecule produced by the method of claim 19; and
b. a substrate to allow for combining said at least one biomolecule with a blood sample from a subject.

61. A kit for the diagnosis of a disorder, disease, or infection comprising
a. at least one Class II MHC tetramer produced by the method of claim 33; and
b. a substrate to allow for combining said at least one Class II MHC tetramer with a blood sample from a subject.

62. A kit for the diagnosis of a disorder, disease, or infection comprising
a. at least one Class II MHC molecule;
b. at least one biomolecule produced by the method of claim 33;
c. at least one tag; and
d. a substrate to allow for combining said at least one Class II MHC tetramer with a blood sample from a subject.

63. The method of claim 18, wherein the mammal is a human.

64. The method of claim 32, wherein the mammal is a human.

65. The method of claim 47, wherein the mammal is a human.

The embodiments are further described in the following examples, which do not limit the scope of the invention described in the claims.

### EXAMPLES

### Example 1

### Materials and Methods

Systems Compiled.

Nineteen systems of CD4+ T-cell immune response mapping studies were collected. Table 1 provides the details of each antigen and citation to the experimental study. In general, each mapping study provides a quantitative profile of immunogenicity for each of the peptides in the set that was tested. For these experiments, epitopes characterized in the literature as ground truth were used, listed in Table 1, Antigen column. For data gathered for the first time, the Wilcoxon signed-rank test was used to determine epitopes.

To apply the presently-disclosed antigen processing-based prediction method, crystallographic B-factors, solvent-accessible surface area, an estimate of local instability in the antigen structure (COREX residue stabilities) and a statistic of evolutionary sequence divergence (Shannon sequence entropy) are utilized. Each parameter provides a quantitative measure of the local conformational flexibility and therefore the likelihood of proteolysis at any particular position in the protein. All antigen structures considered in these experiments were solved using X-ray crystallography with the exception of Bet v, which was solved by NMR. B-factors are a measure of local conformational disorder, which is an indication of how easily the structure may be deformed for binding in a protease active site. Solvent accessible area quantifies accessibility to proteases as well as local disorder.

COREX provides a score of the probability of unfolding at each amino acid and has been validated by correlation with hydrogen-deuterium exchange protection NMR experiments. Hydrogen exchange occurs on much longer timescales than the conformational fluctuations that are captured by B-factors, and therefore it provides complementary information about the likelihood of proteolysis. Sequence entropy is correlated with solvent-accessible surface area, and it provides information on protein segments that were not present in the crystallographic/NMR structure. For this analysis, backbone amide-nitrogen B-factors were extracted from the PDB entries given in Table 1. Likewise, average root-mean-square deviations for the backbone amide nitrogens were extracted from the PDB entry for Bet v. Solvent-accessible surface area was calculated with the crystallographic or NMR structures using Molmol. The COREX/BEST score was computed using the provided web interface. For analysis of sequence entropy, all Swiss-Prot entries having at least 50% identity to the target protein were aligned using ClustalW, and then the Shannon sequence entropy was calculated using BioEdit.

Algorithm.

The input to the presently-disclosed algorithm is the antigen sequence and the four sources of conformational stability data mentioned above: crystallographic B-factors, solvent-accessible surface area, COREX residue stabilities and sequence conservation. The data is preprocessed to register the antigen sequence to the structure. As a PDB file corresponding to the antigen sequence can have missing residues, gaps are addressed by assigning default values for each data type that indicate a minimum level of stability. For example, a missing B-factor would be set to be the maximum B-factor value observed in the PDB file. Then, as a preprocessing step to smooth the data, a windowed average is taken across each system using a window size of 15 residues.

After smoothing the data, z-scores are computed for every residue with respect to each system. The z-scores for each type of data are used as independent observations of antigen conformational stability. Using the analog of Fisher's method for combining test statistics, z-scores are combined to obtain an aggregate z-score for each residue of the antigen. This aggregate z-score characterizes the extent to which the sources of structural data “agree” that the antigen is conformationally unstable at a given position

To describe the construction of our epitope likelihood score, any residue with an aggregate z-score greater than zero is deemed stable and all other residues unstable. To construct the epitope likelihood score, the epitope likelihood of any residue in an unstable region is set to zero. Stable residues adopt their z-score as their epitope likelihood. Then, likelihoods in regions of the protein that transition from unstable to stable or vice versa are selectively upweighted. For example, in an unstable region with a C-terminally adjacent stable region, the epitope likelihoods for first third of the stable region are first magnified by a factor of three. Then, epitope likelihoods are assigned by linearly interpolating from the midpoint of the unstable region to the midpoint of the upweighted portion of the C-terminal flanking stable region. This same upweighting method can be applied to the N-terminal flank or to both the C-terminal and N-terminal flanks of an unstable region. In the results presented here, single-allele systems made use of C-terminal weighting only, whereas multi-allele human systems made use of weighting on both flanks of unstable regions.

MHC Binding Score Prediction.

For each of the antigens above, MHC binding affinity was computed to use as an alternate analysis of predicted immunogenicity. NetMHC-II was used to perform prediction for each antigen sequence. The resulting prediction score (the 1−log 50 K(aff) entry in the output) was used as an epitope likelihood score IEDB also has a set of tools, among which are the NetMHC-II server itself. NetMHC-II was chosen for flexibility as it allows the selection of individual peptide lengths used in the peptide mapping assays for each antigen, which varied in length from between 15 amino acids up to 19 amino acids

Performance Criteria and Evaluation.

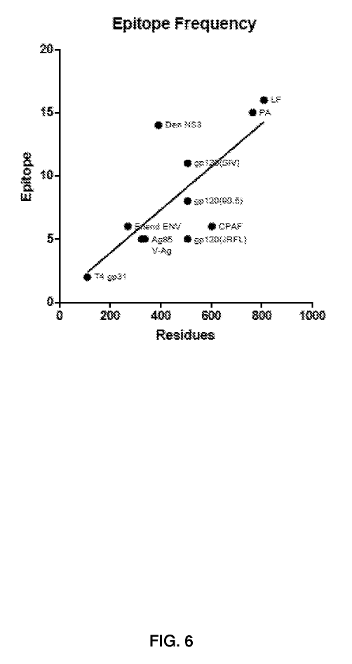

Two metrics were used for evaluating predictive performance with respect to a given a set of epitopes determined experimentally (e.g., with an assay for T-cell proliferation or IFN-γ ELISPOT). First, the number of epitopes recovered by the 90th and 80th percentile scores for a given method of prediction, as shown in Table 1, are considered. For each threshold, the performance of a method is classified based on the number of correctly-identified epitopes. These results provide an evaluation of how effective a particular method is in a real-world setting where a set of peptides must be chosen for testing. To establish baseline performance, both MHC binding-based and antigen processing-based prediction results are compared to the expected number of epitopes at a given threshold, as shown in FIG. 6.) In practice, this baseline approach would correspond to choosing peptides at random with a probability based on an estimated epitope frequency.

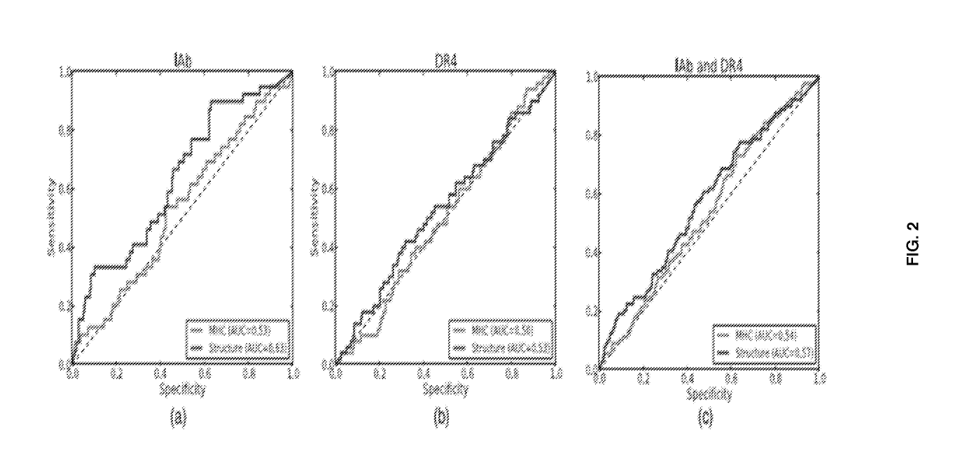

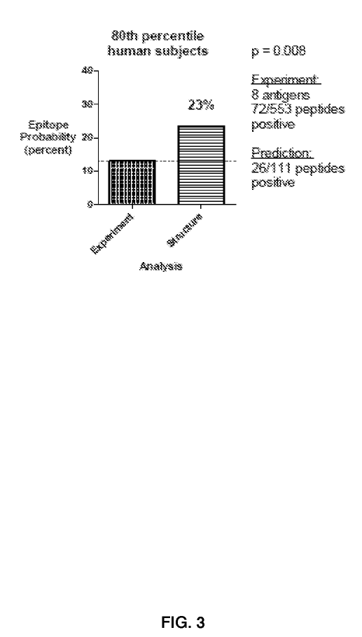

In prior work, evaluation of MHC binding performance has typically been characterized by the area under the receiver-operator characteristic (ROC) curve. As above, for these evaluations, peptides have an underlying classification as a binder or non-binder (determined experimentally) and any method for binding prediction is rated as to its effectiveness in predicting the underlying classification. In the present approach, epitope likelihood is computed on a per antigen basis, and to compare predictions across antigens, scores are normalized to be in the range [0, 1]. Predictions for a collection of peptides spanning multiple antigens can thus be evaluated in the same manner as MHC binding. ROC curves were computed in this way for epitope prediction in both single-allele systems, shown in FIG. 2) and humans, shown in FIG. 3(b).

### Results and Discussion

A total of 19 systems in which epitopes were experimentally determined were considered. For each single-allele system, the performance of the present algorithm was compared to a popular sequence-based method, NetMHC-II. For multiple allele systems, MHC binding methods can only be used when the alleles (or allele distribution) of the population is known. This information was not available for the systems considered herein and thus no MHC binding-based prediction was possible.

TABLE 1
Enrichment results for individual systems. For each system, the number of epitopes
recovered at the 90th and 80th percentile scoring thresholds for this antigen processing-
based approach are listed, along with the standard sequence-based predictions. For human
data, sequence-based methods are difficult to apply since HLA genotypes are in general not
determined for human subjects.

| Allele | Antigen | PDB ID | # epitopes | # peptides | Structure 90% | Structure 80% | MHC 90% | MHC 80% |
|---|---|---|---|---|---|---|---|---|
| I-Ab | HIV JRFL [1] | 3JWO | 5 | 46 | 1 | 2 | 1 | 2 |
| | HIV gpl20 [20] | 1GCI | 8 | 47 | 2 | 2 | 1 | 2 |
| | SIV gpl20 (Nguyen, unpublished) | 3FUS | 11 | 50 | 1 | 3 | 0 | 2 |
| | Y. pestls V-antigen (Charles, unpublished) | 1RGF | 5 | 53 | 2 | 2 | 1 | 2 |
| | Tuberculosis antigen 85A [13] | 1SFR | 5 | 28 | 1 | 2 | 0 | 0 |
| | Friend virus env [25] | 1AOL | 6 | 20 | 2 | 4 | 2 | 3 |
| DR4 | Chlamydia CPAF [21] | 3DJA | 27 | 63 | 2 | 2 | 0 | 2 |
| | Anthrax protective antigen [40] | 1ACC | 15 | 74 | 2 | 4 | 0 | 0 |
| | Anthrax lethal factor [40] | 1J7N-B | 16 | 89 | 2 | 4 | 3 | 3 |
| | Dengue env [30] | 1UZC-A | 14 | 26 | 1 | 4 | 1 | 2 |
| Human | Tuberculosis antigen 85A [36] | 1FON | 14 | 28 | 2 | 4 | — | — |
| | Birch pollen Bet v [14] | 1BTV (NMR) | 5 | 50 | 2 | 2 | — | — |
| | Hepatitis ns3 [10] | 1CUI | 7 | 45 | 1 | 2 | — | — |
| | M. leprac HSP70 [2] | 2V7Y | 6 | 60 | 0 | 3 | — | — |
| | Tick-borne encephalitis protein E [37] | 1SVB | 12 | 121 | 2 | 4 | — | — |
| | Telanus toxoid [15] | 1DLL | 10 | 51 | 2 | 4 | — | — |
| | Wasp Ves [4] | 1QNX | 7 | 65 | 0 | 2 | — | — |
| | H. pylori urease [42] | 1E9Z-B | 2 | 92 | 1 | 1 | — | — |
| | Adenovirus serotype 5 [9] | 3TG7 | 16 | 133 | 4 | 7 | — | — |

The top-scoring peptides at the 80th and 90th percentile thresholds for each method are considered and the number of experimentally determined epitopes that are recovered is reported in Table 1. As a baseline comparison of performance, both methods are compared to the predictive power of the naive random approach, in which a peptide is predicted to be an epitope with a probability equal to the epitope frequency for that antigen, as shown in FIG. 6.

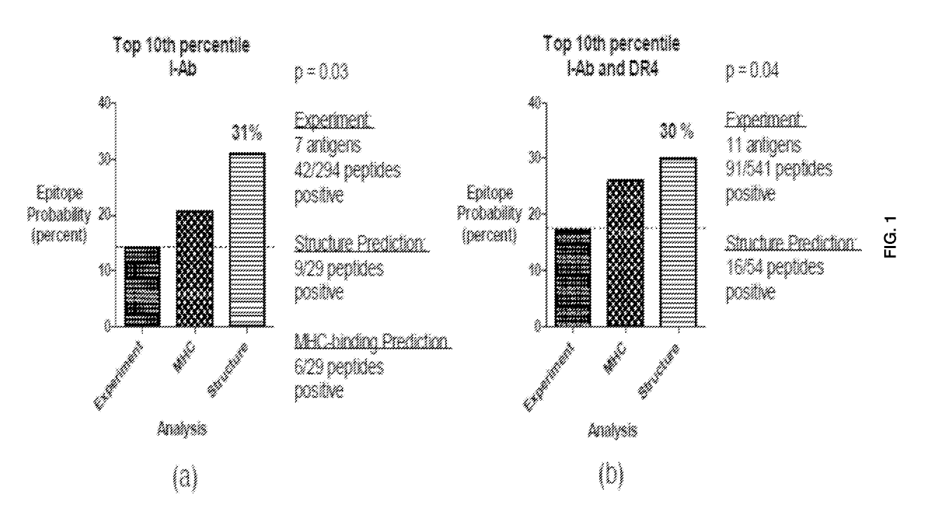

Overall, MHC binding-based epitope prediction did not perform significantly better than random at the 90th percentile threshold. This result is somewhat surprising given the overall effectiveness of predicting the binding affinity of a peptide to a given MHC allele. In contrast, epitope prediction based on conformational stability achieves significantly better prediction than random for single-allele systems, as shown by FIG. 1. For multiple-allele systems, the present algorithm achieves significantly better prediction than random at the 80th percentile threshold, as shown in FIG. 3(a). The 80th and 90th percentile thresholds were chosen using a rationale based roughly on epitope frequency, but the same results of predictive power are evident in ROC curves. For single-allele data, shown in FIG. 2, the present algorithm achieves an AUC that is significantly better than random, while MHC binding-based prediction does not. For multiple-allele systems, shown in FIG. 3(b), this algorithm achieves an AUC that is significantly better than random as well.

LcrV in C57BL/6 Mice.

The LcrV gene-product of Yersinia pestis (the causative agent for plague) is a candidate for use as a subunit vaccine, most commonly as a fusion protein composed of LcrV fused to the C-terminus of the F1 capsular protein. One study has reported MHC-II-restricted epitopes of LcrV in C57BL/6 mice, following immunization by subcutaneous injection of recombinant F1-V emulsified in Freunds adjuvant, a mixture of oil, water, and mycobacterial extract. For the present work, the native conformation of the F1-V was maintained by administering the F1-V intranasally with a mucosal adjuvant (mutant R192G heat labile toxin) in aqueous buffer.

Epitopes were identified by restimulation of splenocytes using individual 17-mer peptides from a set of 53 spanning LcrV in six-residue steps. A response was scored positive if splenocyte proliferation exceeded that of unstimulated controls, as reported by the Wilcoxon signed rank test. Five peptides were identified as containing epitopes in C57BL/6 mice: peptides 19, 20, 28, 29, and 51. The earlier mapping study found epitopes at peptides 13, 18, and 28. Thus, the present study confirmed the epitope at 28, identified neighboring epitopes at 19, 20, and 29, and found a new epitope at 51, but did not find an epitope at 13.

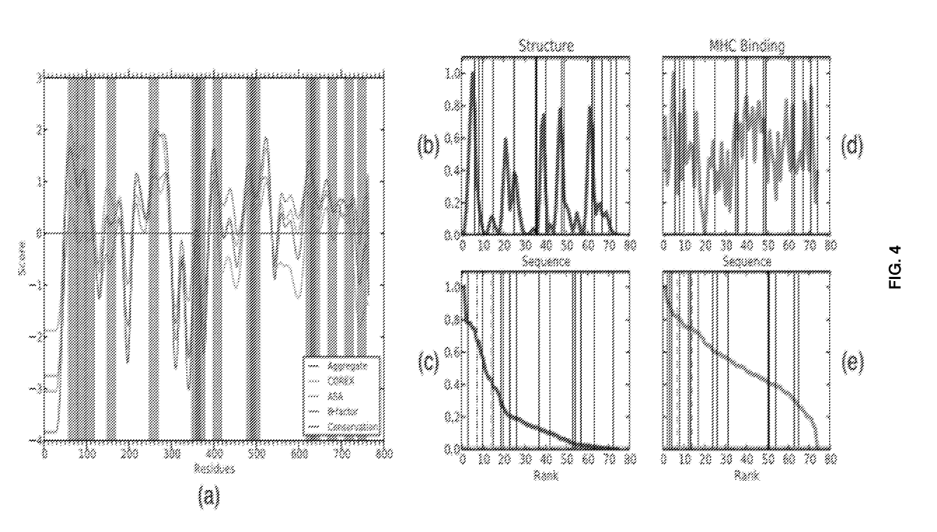

The analysis of conformational stability in LcrV identified 7 major dips in stability that are likely to provide sites for proteolytic processing, shown in FIG. 4(a). Immunogenicity was most strongly predicted on two large transitions from low-to-high stability, spanning peptides 18, 19, 20 and peptides 48, 49, as shown in FIG. 4(b). All three peptides of the first group correspond to the epitopes 18 and 19 observed in C57BL/6 mice. Peptide 20 also was reported by direct submission to the IEDB to be an epitope in immunized HLA-DQ8-transgenic mice (IEDB epitope ID 32126).

Four of the observed epitopes (13, 28, 29, and 51) were not among the 90th percentile of peptides predicted to be immunogenic on the basis of conformational stability, but they still may derive immunogenicity from mechanisms of antigen processing. Epitope 51 adjoins (and shares 5 residues) with the predicted-epitope 49. Peptide 28 appeared in the 75th-percentile and in the third-highest peak of predicted immunogenicity, as shown in FIG. 4(b, c). Thus, it may be necessary to dip into lower percentiles in order to capture all of the dominant epitopes. By concentrating on the 90th percentile, too many epitopes in a single immunogenic segment may be predicted, and epitopes in other immunogenic segments may be missed.

The epitopes observed in C57BL/6 mice form clusters with neighboring epitopes that were observed in other mouse strains. Three of the epitopes observed in C57BL/6 mice (peptides 18, 28 and 29) overlap epitopes that were observed in LcrV-immunized BALB/c mice) or transgenic mice that express a human MHC-II (HLA-DR1 or HLA-DQ8.) Peptide 51 adjoins peptide 48, which was an epitope in LcrV-immunized HLA-DR1-transgenic mice (IEDB epitope ID 9326). Each of these epitope clusters (18-20, 28-29, and 48-51) coincides with a peak of epitope likelihood revealed by analysis of conformational stability, seen in FIG. 4(b).

The NetMHC-II server predicts affinities for peptides, and identifies them as having “strong” or “weak” binding, based on their rank within the hierarchy of a large pool of reference peptides. None of the 53 LcrV peptides was classified as “strong” binding. Peptides 17 and 18 were identified as “weak” binding. Since this threshold may still be too stringent, peptides in the 90th percentile of predicted MHC-II affinity were identified within LcrV, yielding peptides 13, 17, 18, 28, and 45. Peptides 13, 18, and 28 were identified as epitopes in the immunized C57BL/6 mice, although 13 and 18 were observed only in the earlier study that formulated F1-V in Freunds adjuvant.

Summarizing results for LcrV, seven T-helper epitopes were observed experimentally; stability/flexibility successfully predicted three epitopes (peptides 18, 19 and 20), and MHC-binding successfully predicted three epitopes (peptide 13, 18, and 28).

Anthrax protective antigen in HLA-DR4-transgenic mice. The 735-residue anthrax protective antigen (PA) is a component of the anthrax toxin and a candidate for vaccination against infection by Bacillus anthracis. Altmann et al. mapped T-helper epitopes in PA-immunized HLA-DR4-transgenic mice using a complete set of 73 overlapping 20-mer peptides. Fifteen helper T-cell epitopes were reported in the Immune Epitope Database.

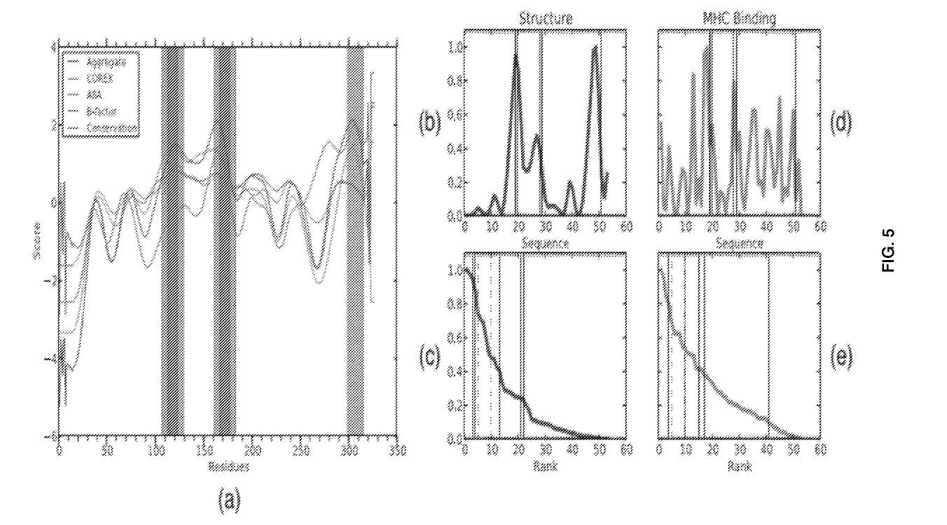

The analysis of stability shown here reveals 6 or 7 large peaks of epitope probability, corresponding to large transitions from low-to-high stability, shown by FIG. 5(a, b). Peptides in the 90th percentile of epitope likelihood include the experimentally-observed epitopes 6 and 62. The 90th percentile also includes peptides 39 and 47, which overlap epitopes 40 and 48. These four epitopes (peptides 6, 62, 40, 48) lie just C-terminal to peaks of epitope probability, suggesting that the present approach correctly predicts epitopes in the region but that epitope likelihood should be shifted or broadened. In spite of the prediction missing nearby epitopes, it was close enough to correctly predict two epitopes within the seven most highly scored (90th percentile) peptides, shown in FIG. 5(c).

Analysis of predicted HLA-DR4 binding with NetMHC-II yielded four “strong binding” peptides, three of which were epitopes in the HLA-DR4 transgenic mice. No additional epitopes were found in the 90th percentile of HLA-DR4 binding. Thus, for anthrax PA, antigen processing-based prediction correctly identifies two epitopes and MHC-binding correctly identifies three epitopes within the respective 90th percentiles of predicted peptides.

Overall performance for single-allele systems. Epitope-predictions based on stability and based on MHC-peptide binding were evaluated for accuracy in the aggregated epitope-mapping results obtained in immunized mice. Each experimental map was generated with a scan for T-cell responses using an overlapping series of peptides that spanned the complete antigen. The input requirements for the epitope predictions limit the number of epitope-mapping studies that may be used for the evaluation. For predictions based on conformational stability, the high-resolution structure of the antigen must have been solved by X-ray crystallography or nuclear magnetic resonance. For predictions based on MHC binding, the T-helper epitopes must have been mapped in mice that have a single well-characterized MHC-II protein. The evaluation also excluded studies on mammalian antigens, for which some epitopes may have been suppressed by negative selection. Epitope maps of influenza antigens were excluded because epitope placement is exceptional, in that epitopes consistently appear on the N-terminal flanks of flexible sites, possibly due to viral modifications to antigen processing mechanisms.

These limitations reduced the available experimental systems to C57BL/6 mice (44 epitopes in 7 antigens) and HLA-DR4-transgenic mice (72 epitopes in 4 antigens). Several epitope-mapping studies in BALB/c mice have been reported, but the number of epitopes discovered is low (11 epitopes in 5 antigens). The BALB/c strain also has two MHCII proteins (I-Ad and I-Ed), which potentially complicated the MHCII-binding prediction and the interpretation of mapping data. Thus, results from BALB/c mice were not included. The several mapping studies in HLA-DR1- and HLA-DQ8-transgenic mice yielded only a small number of epitopes, and thus they were not included. For comparison, the IEDB lists 377 I-Ab-restricted epitopes in 146 non-mammalian antigens. Most of these epitopes were not included in the present study because the antigen's high-resolution structure was not available (e.g., a membrane protein or intrinsically disordered protein) or because the T-cell responses were not scanned with a complete set of overlapping peptides. For incomplete scans, the test peptides were usually selected by the predicted ability to bind the I-Ab molecule.

Accuracy of epitope prediction in C57BL/6 mice was evaluated by comparing the 90th percentile of predicted epitopes to the experimentally discovered epitopes, shown in FIG. 1(a). Using the present antigen processing-based method for epitope prediction, 31% of peptides scoring in the 90th percentile of predicted immunogenicity were actually observed as epitopes. This is a significant improvement over random peptide selection (p=0.04) and compares favorably with the prediction based on peptide-binding to the I-Ab MHC-II, for which 28% of peptides scoring in the 90th percentile of I-Ab binding were observed as epitopes.

In HLA-DR4 transgenic mice, epitopes were discovered at a rate of 21% of the test peptides. Epitope prediction at the 90th percentile using antigen processing-based prediction and HLA-DR4 binding-based prediction yielded accuracies of only 28% and 32%, respectively, which was not significantly greater than expected for a random selection of peptides.

For the combination of antigens in C57BL/6 and HLA-DR4 transgenic mice, 30% of peptides predicted by stability/flexibility were observed as epitopes, shown in FIG. 1(a, b), which is significantly greater (p=0.04) than for a random selection of peptides (17%). Likewise, 30% of peptides predicted by MHC-II binding affinities were observed as epitopes, shown in FIG. 1(b).

Bet v in birch-allergic human subjects. T-helper epitope maps from human populations are difficult to interpret due to genetic heterogeneity of class II MHC proteins. Scores of different class II alleles are represented in some populations, and individuals may express six different alleles. For most epitopes, the restricting MHC allele is not known or multiple alleles contribute to presentation. In spite of multiple sources of variability, strong epitope dominance is still observed in the human immune responses.

Bet v of birch pollen is one of the most thoroughly studied allergens. Bohle et al. mapped the T-helper epitopes of the 159-residue Bet v in a group of 57 birch-allergic subjects using a set of 50 overlapping 12-mer peptides. As expected for a heterogeneous population, the T-helper epitopes were distributed over most of the protein. Forty-three peptides obtained a response from at least one subject, and 35 peptides obtained a response from at least two subjects. Here, the immunodominant epitopes are defined as the five most frequently immunogenic peptides (90th percentile), each of which obtained a response from at least 11 subjects (peptides 2, 5, 7, 38, and 48). The single most frequently immunogenic epitope (peptide 48) obtained a response in 32 subjects

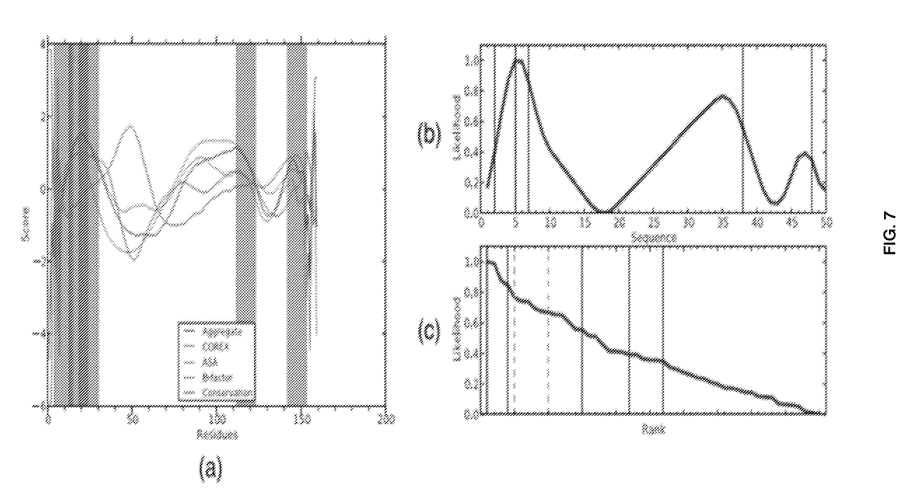

The analysis of conformational stability in Bet v found two major dips in stability and predicted epitopes in the transitions from low-to-high stability on both sides of the stability minima, resulting in three peaks of predicted immunogenicity, shown in FIG. 7(a, b). This equal weighting of N- and C-terminal flanks was adopted on the basis of a previous analysis of immune responses in outbred populations to nine different antigens/allergens. Four Bet v peptides in the 90th percentile of predicted immunogenicity were located in the first major peak, and they include the observed epitopes 5 and 7. Although having generally lower predicted immunogenicity, the second and third peaks also included at least one of the observed epitopes.

The fact that the highly dominant peptide 48 coincided with the smallest of the three peaks of predicted immunogenicity in Bet v suggests that that the prediction failed to capture an important aspect of the mechanism for dominance. The missing element may involve the ease with which a proteolytic fragment dissociates from the otherwise intact antigen. The current algorithm assigns high immunogenicity to peptides that are located within stable segments adjacent to highly flexible segments. This weighting recognizes the probability of initial cleavage in the flexible site and assigns immunogenicity to the adjacent stable segment. It does not account for the requirement that the MHC-II protein gain access to the stable segment, which is likely to involve the dissociation of the epitope-containing segment from other stable segments.

The exceptional immunodominance of peptide 48 has been attributed to its early and abundant processing and presentation. Peptides presented by dendritic cells were compared to the proteolytic fragments generated in a time-course of Bet v digestion in lysosomal extracts. The most abundantly-presented peptides corresponded to fragments that were generated only at early time-points (0.5-3 hours). It remains unclear why peptides generated at later time-points (5-24 hours) are poorly presented. The late peptides appear to be in equal or greater abundance compared to the early peptides at the respective time points, and thus proteolytic destruction of late peptides seems to be ruled out. Mechanisms of intracellular traffic may be responsible. During the maturation of dendritic cells, ubiquitination in the C-terminal tails redirects MHCII from the lysosome to the cell surface, and thus the MHCII may not be available to bind peptides that emerge from late stages of antigen processing.

Adenovirus type 5 hexon in HIV-vaccine trial participants. Pre-existing immunity to the adenovirus type 5 (Ad5) has been linked to weaker responses to Ad5-based vaccines. Initial attention focused on the ability of the antibodies to neutralize the vaccine, and this spurred the development of adenoviruses having low prevalence in humans and little antibody crossreactivity. However, the highly conserved T-cell responses to the hexon subunit of Ad5 recently have also been implicated in the weak responses to Ad5 vaccines. Thus, the relationship of structure and T-cell epitope dominance was examined in hexon, a 947-residue capsid protein.

McElrath et al. mapped T-helper epitopes for the Ad5 hexon using 133 overlapping 15-mer peptides with the PBMC of 32 subjects participating in an HIV-vaccine trial. Although approximately half of the subjects were sero-negative for Ad5, most reacted to at least one T-cell epitope, probably because the T-cell epitopes are conserved in other adenoviruses to which the subjects had been exposed (e.g., Ad1 or Ad2). As expected for a heterogeneous population, epitopes were distributed over a large portion (40%) of the hexon sequence. For the present comparison to epitope prediction, the 16 peptides that stimulated a T-cell response from two or more subjects, corresponding to the 88th percentile of the tested peptides, were designated as immunodominant.

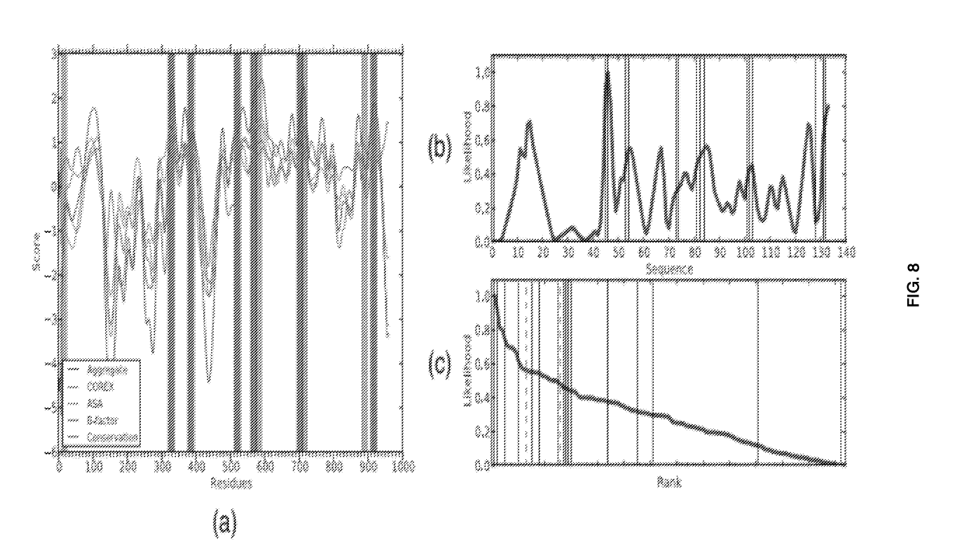

The analysis of hexon conformational stability found at least eight major dips in stability, which gave rise to adjacent peaks of epitope likelihood, shown in FIG. 8(a, b). Peptides in the 90th percentile touched five peaks of epitope probability. Two peaks contained four peptides that match observed epitopes (peptides 45, 46, 131, and 132). Although the epitopes occurred as two pairs of overlapping peptides, it is not clear that each pair should be considered a single epitope because the restricting MHC alleles are unknown. An alternative explanation is that the pairs of epitopes arose from the same abundantly-processed antigen fragments, as suggested by their locations at the transitions from low-to-high stability. Looking at the 80th percentile, the antigen processing-based approach accurately predicts three more epitopes (peptides 54, 82, and 84).

In summary, for Ad5 hexon, 4 of 13 peptides in the 90th percentile and 7 of 26 peptides in the 80th percentile were observed as epitopes. Of the eight transitions from low-to-high stability (peaks of epitope likelihood), four contained epitopes in the vaccine trial participants.

Overall performance for humans. Antigen processing-based epitope prediction was evaluated for the aggregated results of mapping studies performed in human subjects. As noted above for the single-allele studies, the analysis was limited to non-self antigens for which a crystal structure was available and where the epitope-mapping employed a complete series of overlapping peptides. The identification of a discrete set of all epitopes for a given antigen is difficult for systems with multiple MHC alleles because the immunogenicity of some epitopes could be strongly allelle-specific. If the investigators identified a discrete set of epitopes, then these were included as the immunodominant set. If the investigators reported a frequency of response for all peptides, then the peptides of the 90th percentile were included as the immunodominant set. In all, 72 epitopes were identified as immunodominant in 8 antigens. Since the antigens were scanned with a total of 553 peptides, this represents a rate of discovery for immunodominant epitopes of 13%. This is the threshold for useful accuracy of epitope prediction in this set of antigens.

At the 90th percentile, the sample of epitopes was too small for the epitope prediction by stability/flexibility to be significantly greater than that of random selection. However, at the 80th percentile, the accuracy of 23% achieved significance (p=0.008), shown in FIG. 3(a). To gain additional perspective, the receiver-operator characteristic (ROC) taken over all predictions for human alleles was considered, as shown in FIG. 3(b). The receiver-operator characteristic (ROC) curve is a conventional method for evaluating comparing predictive power at all thresholds of prediction. A ROC curve area that is significantly greater than 0.5 indicates predictive power; the presently-disclosed algorithm achieves an area under the curve of 0.64 (p=0.002).

### Example 2

Two different machine learning approaches based on hidden Markov models (HMMs) and position-specific scoring matrices (PSSMs) are proposed. Both of these approaches provide the necessary flexibility to incorporate training data for MHC binding in single-allele systems, and to use only structural data for multiple-allele systems. HMMs only require gathering of sequential dependencies between amino acid positions across the training set, and are computationally efficient to train. PSSMs construct an explicit sequence motif, but require the computationally expensive step of performing a multiple sequence alignment on all peptides in the training set. Structural data will be incorporated into the Gibbs sampling approach of SMM-align [36]. First, peptide contributions to the final motif will be pre-weighted utilizing an aggregate measure of conformational stability per peptide in the training set. Next, conformational stability data will be incorporated into the Gibbs sampler itself so as to bias the search for the motif in a structurally appropriate manner. Finally, matrix methods also use “pseudocounts,” which are correction factors used to smooth or normalize amino acid frequencies that occur in the motif being constructed. Structure-specific amino acid propensities may be used to construct novel pseudocounts for use in the PSSM models.

Two independent training sets will be used to test the process. Performance with respect to each source of structural data will be examined as well as molecular dynamic simulations.

First, the algorithms will be trained on WWII presentation data being collected on very large datasets of human MHC ligand sequences from B lymphocytes using high-throughput mass spectrometry. Data from this study is ideal for training; the initial dataset contains 1,704 peptides representing 1,110 proteins and was collected under a consistent set of conditions. The majority of the work in making this data suitable for use in the embodiments disclosed herein is to identify the correct association with the three-dimensional structure if it has been solved (estimated at 30-40% of total soluble, cytoplasmic antigens, approximately 400 proteins). Each peptide identified by the study will be cross-referenced to its associated antigen to identify the associated antigen structure and compute conformational stability criteria as input to the prediction scheme.

The second data set will come from mining of the Immune Epitope Database (IEDB) [50] for peptides with T-cell response data for available single- and multiple-allele systems. The IEDB contains a significant amount of useful data: for mouse, there are around 15K peptides with experimental measurements (roughly 6K positive, and 9K negative), and about 20K peptides for human (about 11.5K positive and 8500 negative). As above, the majority of the work in making this data suitable for use in the embodiments disclosed herein is in identifying the correct association with the three-dimensional structure if it has been solved.

Data from the mass-spectroscopy sequence will capture MHC II presentation, while the datasets data derived from sources like the IEDB will provide peptide mapping results for CD4+ T-cell response. The latter data provides information downstream of T-cell recognition. Nevertheless both datasets will allow evaluation of the contributions of each source of structural information. An HMM model trained as described above has state transition weights that provide information about the sequential pattern of each structural data that is necessary to best identify an epitope. The PSSM approach is more global and the learned positional weights provide an idea of the relative importance of each source of structural information. By analyzing these contributions it will be possible to identify to what extent each source of information contributes to identification and decide whether modifications to the models (e.g., use higher-order Markov models, modify Gibbs sampling density) are required.

In addition to the above, MD simulations will also be utilized to check that the parameters derived from the learning step (e.g., proteolytic sensitivity) are physically realistic. MD simulations will be performed using NAMD [41] and Amber [52] to characterize the conformational stability of peptides in a given antigen structure in two ways. First, for each antigen appropriate MD runs (with implicit solvent modeling) will be configured on an antigen structure to obtain free energy estimates for the entire structure. The parameters for each antigen will then be set so that it is in an appropriate equilibrium at conditions that accurately reflect the endocytic compartment where MEW binding occurs. Additionally, the addition of entropy-based scores on a per residue basis as a fifth source of structural data for our algorithm will also be explored.

For each of the two training datasets a cross-validation approach will be used for testing and refinement, in which whole antigens are left out for testing. In this way, expected performance on a variety of antigens can be assessed. For each such cross-validation experiment, the performance of both HMM and PSSM approaches will be tested utilizing an enrichment threshold and ROC curves. Performance of these methods will then be compared against a baseline of random peptide sampling, as well as existing epitope prediction methods (e g IEDB tools, NetMHC II) whenever possible.

Predict and Validate CD4+ T-Cell Epitopes for Five Soluble Vaccine Candidates from Each of Two Pathogenic Gram-Negative Bacteria. Salmonella typhimurium and Burkholderia pseudomallei

The predicted epitopes of five proteins (SipA, SopE, SifA, SopA, SipD) from Salmonella typhimurium and five proteins from Burkholderia pseudomallei (BPSL2096, BPSS1492 (BimA), BPSS1512 (TssM), Tuf (Ef-Tu), BPSL2403) will be tested by analyzing CD4+ T-cell immune response in vivo in C57BL/6 mice. These two bacterial organisms were chose because of their biomedical importance: both bacteria are listed as biological risk category B, and are also considered potential bioterror agents because of their infectivity through the respiratory route. Importantly, these particular antigens from each bacterium were chosen because they are vaccine candidates. Epitopes predicted at the 80th percentile threshold by the embodiments disclosed herein and by MHCII binding will be compared with experimentally observed T cell responses.

Groups of mice will be immunized by infection with Salmonella or Burkholderia or by vaccination with pairs of the purified recombinant proteins. The use of both types of exposure is important. Natural infection offers the most relevant exposure, but immune responses to some proteins could be too weak to systematically analyze the epitopes. Immunization with a purified protein potentially increases the strength of response to the protein, but the profile of epitope dominance could be affected by the method of preparation or circumstances of exposure. For epitope testing, peptides representing the 80th percentile by prediction (roughly 6 peptides per antigen) will be obtained from commercial sources. To maintain good sensitivity and reproducibility, proliferative responses will be identified by re-stimulation of splenocytes (3 105 cells per well) with individual peptides (0.4 microgram). After culturing for three days, 100 microliters of supernatant will be removed and archived for analysis of cytokine production in future studies, and replaced with fresh medium containing [3H]thymidine. After another day of culture, cells will be harvested, and the incorporated radioactivity will be measured by scintillation counting. Background proliferation will be determined by the average and standard deviation for proliferation in at least 7 wells of un-stimulated cells from each mouse.

Groups of 20 C57BL/6 and 20 BALB/c mice will be infected with S. typhimurium or B. pseudomallei. After immunity has a chance to develop in each group, blood and spleens will be recovered for measurement of CD4+ T-cell immune responses against two soluble vaccine-candidate proteins. Predicted epitopes from all five soluble vaccine-candidate proteins will be tested in each mouse (approximately 30 peptides). For this step, the total animals used will be 20 mice×2 infections×2 mouse strains, 80 mice.

Coding sequences for the mature forms of vaccine-candidate proteins will be amplified from chromosomal DNA using a 3 primer that encodes a His6 tag and then inserted into a cloning vector suitable for overexpression in E. coli. Recombinant proteins will be purified by nickel-affinity chromatography. Groups of 10 C57BL/6 and 10 BALB/c mice will be immunized by three subcutaneous administrations at two-week intervals of 2 proteins, 10 micrograms each with 10 micro-grams of Detoxified Monophosphoryl Lipid A (MPL) from InvivoGen. This protocol was selected because it resembles vaccination protocols that are advancing toward clinical use [15]. For this step, the total animals used will be 10 mice×5 protein pairs×2 mouse strains, or 100 mice. Mice will be sacrificed one week after the third administration. Immunization will be confirmed by serum antibody titer. CD4+ epitopes will be identifed by splenocyte proliferation as described above.

Cells responding to the single most immunogenic pep-tide from each antigen (5 antigens) will be confirmed as enriched for CD4+, CD44+(antigen-experienced), IFN-+, and not CD8+, compared to cells from naive mice. Three mice will be infected as in Aim 2.1 or uninfected. Splenocytes (2∴|10⁶) will be re-stimulated with a single peptide for 6 hrs (with secretion blocked for the final 5 hrs), treated for staining and fixation, and counted by flow cytometry. Total animals used will be 3 mice×2 mouse strains×3 infections/uninfected, 18 mice.

All publications, patents, and patent applications mentioned herein are incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method for identifying immunogenic epitopes in target polypeptide sequences, comprising:
determining, using a computing device, an epitope likelihood for reach residue in a target polypeptide based at least in part on a sequence and a conformational stability profile for the target polypeptide;
selecting, using the computing device, one or more epitopes from the target polypeptide sequence with an epitope likelihood score above a defined threshold.

2. The method of claim 1, wherein the epitope likelihood indicates a potential of the epitope to bind WIC II.

3. The method of claim 1, wherein determining the epitope likelihood comprises:
registering, by the computing device, the target polypeptide sequence to the conformational stability profile such that each residue in the polypeptide sequence is associated with a set of conformation stability data for that residue;
determining, by the computing device, an aggregate z-score for each residue based on the aggregate conformation stability data for each residue, wherein the aggregate z-score indicates that a residue is stable or unstable;
mapping, by the computing device, regions of conformational stability and instability in the target polypeptide based on the aggregate z-score for each residue;
determining, by the computing device, an epitope likelihood based at least in part on a proximity of an epitope to a midpoint of an unstable region of the target polypeptide.

4. The method of claim 3, wherein determining the epitope likelihood comprises linearly interpolating from a midpoint of an unstable region to a midpoint of a stable region, wherein the epitope likelihood score for each unstable residue is initially set to zero, the epitope likelihood for each stable residue is initially set to the z-score for that residue, and the initial epitope likelihoods are upweighted in regions that transition from unstable to stable or stable to unstable.

5. The method of claim 1, wherein the conformational stability data comprises crystallographic B-factors, solvent-accessible area, COREX residue stabilities.

6. The method of claim 1, wherein a sequence conservation score is used for residues not represented in the conformation stability profile.

7. A method for identifying immunogenic epitopes comprising:
identifying, using a computing device, one or more epitopes within a target polypeptide using one or more classifiers applied to a sequence of the target polypeptide, a conformational stability profile of the target polypeptide, or both.

8. The method of claim 7, wherein the identified epitopes are identified based on a potential to bind to MHC II.

9. The method of claim 7, wherein the classifier is trained on a training set comprising sequences and conformational stability profiles of peptides known to bind or not bind MHC II.

10. The method of claim 7, wherein the conformational stability profile comprises crystallographic B-factor data, solvent-accessible surface area data, COREX residue stabilities data.

11. The method of claim 7, where classifier is derived using supervised or unsupervised machine learning.

12. The method of claim 11, wherein the machine learning is based on a hidden markov model (HMM) or position-specific scoring matrices (PSSMs).

13. The method of claim 12, wherein the machine learning is based on a PSSM, and wherein each peptide in the training set is weighted using an aggregate measure of conformation stability.

14. The method of claim 13, wherein the PSSM comprises a Gibbs sampler, wherein conformational data is incorporated into the Gibb's sampler.

15. The method of claim 1, further comprising preparing one or more compositions comprising the one or more selected epitopes.

16. The method of claim 15, further comprising administering to a subject an effective amount of the one or more compositions.

17. The method of claim 16, wherein the one or more compositions further comprise at least one adjuvant, at least one binder, at least one diluent, at least one excipient, or mixtures thereof, or wherein the one or more compositions are formulated for oral or parenteral administration, or wherein the one or more compositions are formulated for oral administration in the form of a concentrate, a dried powder, a liquid, a capsule, a pellet, or a pill.

18. (canceled)

19. (canceled)

20. The method of claim 16, wherein the subject suffers from an allergy, an autoimmune disorder, an infection, or cancer.

21. A pharmaceutical composition comprising the one or more epitopes identified using the method of claim 1, wherein the composition optionally further comprises at least one adjuvant, at least one binder, at least one diluent, at least one excipient, or mixture thereof.

22. (canceled)

23. The method of claim 7, further comprising preparing one or more compositions comprising the one or more selected epitopes.

24. The method of claim 23, further comprising administering to a subject an effective amount of the one or more compositions.

25. A pharmaceutical composition comprising the one or more epitopes identified using the method of claim 7.
